(19) United States
(12) Patent Application Publication
(10) Pub. No.: US 2023/0140392 A1
(43) Pub. Date: May 4, 2023

(54) OVERMOLDED CONNECTORS AND METHODS OF MAKING THE SAME

(71) Applicant: ENTEGRIS, INC., Billerica, MA (US)

(72) Inventor: David Schroeder, Bloomington, MN (US)

(21) Appl. No.: 17/976,005

(22) Filed: Oct. 28, 2022

Related U.S. Application Data
(60) Provisional application No. 63/274,357, filed on Nov. 1, 2021.

(51) Int. Cl. F16L 41/03; F16L 57/00; F16L 41/02

(52) U.S. Cl. CPC F16L 41/03; F16L 57/00; F16L 41/023

(57) ABSTRACT

A fluid connection assembly that includes a plurality of tubes having a first open end and a second open end opposite the first open end and a plurality of connectors. Each of the connectors include at least two connector portions, in which each of the at least two connector portions extend from a center of the connector and have a first end connected to the center of the connector and a second end connected to the second open end of one of the plurality of tubes. The connector portions are continuously formed with the tube and the center of the connector and the connector portions are fluidly connected together.

### PRIORITY

This disclosure claims priority to U.S. provisional patent application No. 63/274,357 with a filing date of Nov. 1, 2021. The priority document is incorporated by reference herein.

### FIELD

This disclosure is directed to a fluid connection assembly for transferring fluids, such as from chemical and/or biological processes, through a number of different tubing, couplings, and storage containers.

### BACKGROUND

Chemical and/or biological processes can utilize or produce process materials that are stored within storage containers, such as bags, containing pharmaceutical or biological fluids, bioprocess bags, and the like. Tubing or other types of coupling and connectors may be utilized to supply the process material and/or reactants into the storage container. The process materials may need to be frozen or otherwise kept at low temperatures within the storage container. Tubing or other types of coupling and connectors may then be utilized to remove and/or transfer the process material from the storage container.

### SUMMARY

Some embodiments of a fluid connection assembly include a plurality of tubes and a plurality of connectors, in which each of the connectors include at least two connector portions. The connector portions extend from a center of the connector and have a first end connected to the center of the connector and a second end connected to the tube(s). The connector portions are continuously formed with the tube and the center of the connector, so that the connector portions are fluidly connected together. Each of the connector portions are conically tapered in which an outer diameter of the first end at the center of the connector is larger than an outer dimeter of the second end of the connector portion in a way such that the connector portion is flexible. In some embodiments, the connector portion(s) include strain relief portions provided along an outer surface of the connector portion. In some embodiments, the strain relief portions include a plurality of rib sections provided in parallel arrangement along a length direction of the connector portion, in which a rib section provided nearer the center of the connector has an outer diameter larger than a rib section provided at the second end of the connector portion. In other embodiments, the strain relief portions include spiral rib sections provided along a length direction of the connector portion, in which a first end of the spiral rib section provided nearer the center of the connector is provided at the first end of the connector portion having an outer diameter larger than a second end of the spiral rib section provided at the second end of the connector portion. In yet other embodiments, the strain relief portions the include segmented core portions removed from the outer surface of the connector portion. It is appreciated that such structures allow the connector and connector portions to be flexible.

Other embodiments may include a method of manufacturing a fluid connection assembly that includes a connector having connector portions that are continuously formed with tubes. In some embodiments, the continuous formation of the connector portions and tubes is performed by overmolding the connector portion with the tube so that the connector portion is fluidly connected with the center of the connector and the tube.

The details of one or more embodiments of the disclosure are set forth in the accompanying drawings and the description, and from the claims.

### BRIEF DESCRIPTION OF DRAWINGS

References are made to the accompanying drawings that form a part of this disclosure, and which illustrate embodiments in which the systems and methods described in this specification can be practiced.

FIG. 1 is a schematic view of a fluid connection assembly according to an embodiment.

FIGS. 2A-2D are front, cross-sectional, and perspective views of the connector of the fluid connection assembly in FIG. 1.

FIGS. 3A-3B are a front view and a cross-sectional view of another embodiment of the strain relief portion of the connector.

FIGS. 4A-4B are a front view and a cross-sectional view of another embodiment of the strain relief portion of the connector.

FIGS. 4C-4D are a front view and a cross-sectional view of another embodiment of the strain relief portion of the connector.

FIGS. 4E-4F are a front view and a cross-sectional view of another embodiment of the strain relief portion of the connector.

FIGS. 4G-4H are a front view and a cross-sectional view of another embodiment of the strain relief portion of the connector.

FIGS. 5A-5B are a front view and a cross-sectional view of another embodiment of the shape of the connector.

FIGS. 5C-5D are a front view and a cross-sectional view of another embodiment of the shape of the connector.

FIG. 5E is a front view of another embodiment of the shape of the connector.

FIG. 5F is a perspective view of a spine assembly and connector of at least one embodiment.

FIG. 6 is flow chart showing a method of manufacturing a fluid connection assembly having a connector.

Like numbers represent like features.

### DETAILED DESCRIPTION

This disclosure relates generally to a fluid connection assembly for transferring a fluid. More specifically, the disclosure relates to a fluid connection assembly that includes a plurality of tubes and a plurality of connectors for the transferring, e.g., filling and removal, of fluids from storage containers. While the fluids related to chemical and/or biological processes are discussed below, it is appreciated that such discussion is not intended to limit the scope of the invention, but provided as embodiments thereof.

Some chemical and/or biological processes utilize or produce process materials that are stored within storage containers, such as bags, containing pharmaceutical or biological fluids. The pharmaceutical or biological fluids may also need to be frozen or otherwise kept at low temperatures within the storage container. It is appreciated that a fluid includes, but is not limited to, a substance that flows or deforms when a shear stress is applied. A fluid can include, for example, a liquid.

Fluid connection assemblies that include tubing and/or other types of coupling and connectors may be utilized to supply and/or remove the pharmaceutical or biological fluids into/from the multiple storage containers, e.g., bag assemblies. For example, connectors are used to connect tubes with the bag assemblies to form spine assemblies for the transferring of the pharmaceutical or biological fluid to/from the multiple storage containers.

It was observed, however, that prior art connectors used rigid connectors, e.g., not able to be bent, that included a hose barb and a mechanical connector, e.g., tri-clamp connection system, for connecting the rigid connector to the tubing. Not only did such prior art connectors create gaps internally between the tubing and the rigid connector, which can form pockets that accumulate fluid and lead to contamination and breakage of cells in the fluid, it was observed that when the tubing connected to the rigid connector was bent, interruptions in the flow path were created, e.g., due to a bending of the tubing creating a kink in the tubing and/or creating an ovular internal shape affecting the flow path. In order to overcome the restriction of the flow path, the process pressure was typically increased. Such increase of pressure not only resulted in different supply pressures being provided to the bag assemblies, which resulted in overfilling and under filling of the bag assemblies, the increased pressure also resulted in failures of the tubing, connectors, mechanical connector, and/or the bag assemblies causing a leakage of the pharmaceutical or biological fluid. Thus, the prior art connectors not only created restrictions in the flow path, the connectors also provided increased potential points of failure, e.g., higher pressures and mechanical connectors that resulted in leakage of the fluids due to pressure differentials in the spine assemblies.

It was also observed that the rigid connectors and spine assembles of the prior art assemblies that used mechanical connectors, when stored at cryogenic temperatures of −190° C. or lower, the ultra-cold temperature caused a leakage in the spine assemblies that allowed ingress of the cryogenic fluid, e.g., liquid nitrogen or similar, into the storage containers or leakage of the pharmaceutical or biological fluid. Without wishing to be bound by theory, it is understood that upon introduction of the spine assemblies in the cryogenic system, the spine assemblies, tubing, and/or rigid connectors, which can be made of different materials having different thicknesses and having different coefficients of thermal expansion (and contraction), contract at different rates and/or have different thermal properties, e.g., rigidity/flexibility, especially at the ultra-cold cryogenic temperatures. Thus, when the spine assemblies, tubes, and the connectors are introduced into the cryogenic system, due to the different contraction rates and/or different thermal properties, the coupling between the spine assemblies, e.g., the tubes, the mechanical connectors, and connectors, fails, which allows ingress of the nitrogen, either in liquid or gas form, into the storage container or leakage of the pharmaceutical or biological fluid.

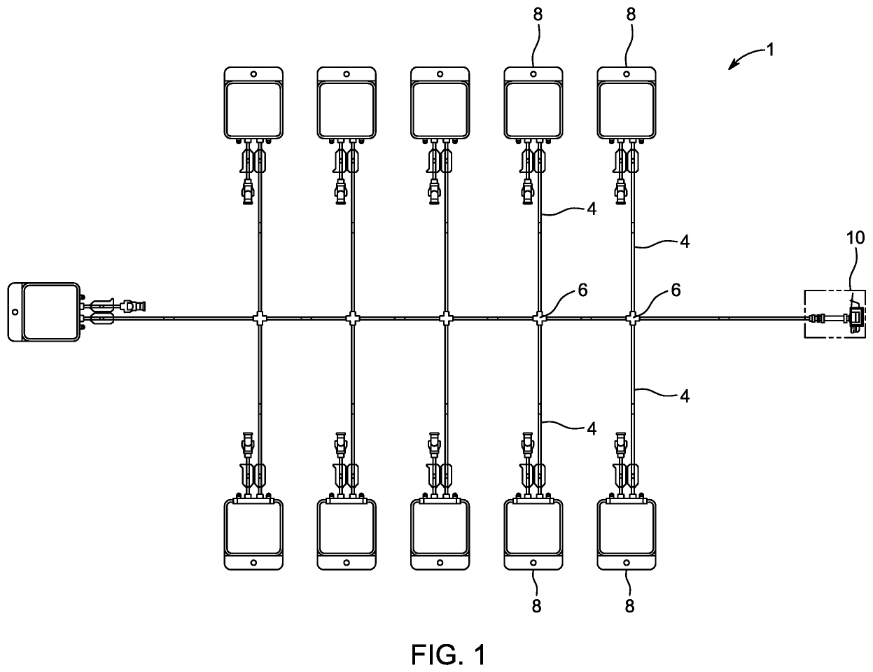

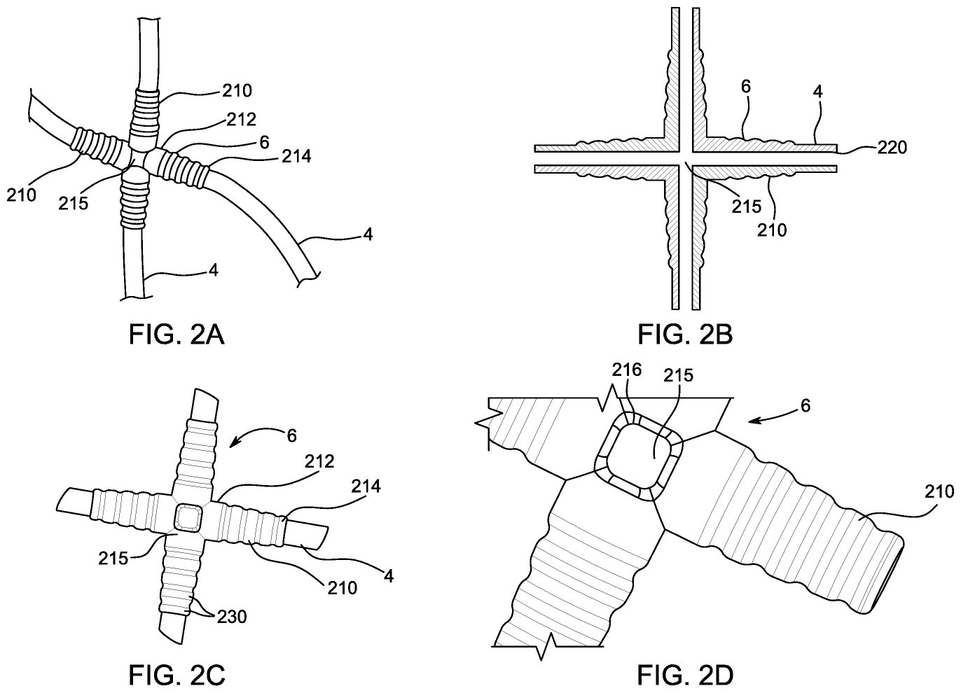

Referring to FIGS. 1-2C, an embodiment of a fluid connection assembly 1 that overcomes the deficiencies of the prior art is shown. The fluid connection assembly 1 includes a plurality of tubes 4 and a plurality of connectors 6, which together form spine assemblies, that are used to connect bag assemblies 8 for the transferring of pharmaceutical or biological fluids from/to processing equipment 10. The plurality of tubes includes tubes having an inner diameter between ⅛ inch and 1 inch with an outer diameter between ¼ inch and 1¼ inch or combination thereof. The bag assemblies can be low-temperature fluid storage containers or similar storage container for the storage of pharmaceutical or biological fluids.

The fluid connection assembly 1 is a pressurized system for the transferring of the pharmaceutical or biological fluid. The fluid connection assembly 1 can be used to distribute the pharmaceutical or biological fluid equally to the bag assemblies. In other embodiments, the pharmaceutical or biological fluid can be distributed in different amounts to the bag assemblies which is dependent on the needs of the user, e.g., different sized bag assemblies. It is appreciated in some embodiments, instead of the fluid connection assembly being used in a pressurized system, negative pressure can be used to transfer the pharmaceutical or biological fluid from the bag assemblies.

FIGS. 2A-2D illustrate an embodiment of one of the plurality of connectors. As seen in FIG. 2A, the connector 6 includes a plurality of connector portions 210 that extend from a center 215 of the connector 6. The plurality of connector portions includes at least two connector portions, but can include between 3 and 10 connector portions, and preferably includes four connector portions. A first end 212 of the connector portion 210 is continuously formed with the center 215 of the connector 6, while a second end 214 is continuously formed with one end of the tube 4. The tube 4 can have its other end connected to at least one of the following: bag assemblies 8, processing equipment 10, another connector 6, or other processing device for pharmaceutical or biological fluids.

The plurality of tubes 4 and the connectors 6 can be formed from a variety of thermoplastic polymers and/or thermoset elastomers so that the tubes and connectors are flexible. For example, the tube 4 and/or the connector 6 can be formed from thermoplastic polymer selected from the group consisting of fluoropolymers, polyurethanes, vulcanizate, flexible polyvinyl chloride (PVC), thermoplastic elastomer (TPE), high-density polyethylene (HDPE), ethylene-vinyl acetate (EVA), co-polymer/polyolefin, high-impact polystyrene (HIPS), polypropylene (PP), acrylonitrile butadiene styrene (ABS), polytetrafluoroethylene (PTFE, or ETFE), or blend of the same, or a thermoset elastomer, such as liquid silicone rubber (LSR) or blend of the same. Thus, the tubes and/or connectors are made from a material that remains flexible even at cryogenic temperatures, e.g., −196° C. The tube and connector can be made from the same material or a different material, but are made generally from material that are materially compatible, e.g., the tube and connector have similar or the same coefficients of thermal expansion/contraction, similar melting temperatures and flow characteristics, the same chemical resistance or compatibility, and/or other properties required by an application for a fluid connection assembly, such as UV blocking and the like. The tube and/or connector can also be made of material that is relatively inert, e.g., does not leach or significantly absorb the pharmaceutical or biological fluid, and non-reactive.

FIG. 2B shows a cross-sectional view of the connector 6. The connector portion 210 is continuously formed with the tube so that an internal flow path 220 is continuously formed between the connector portions 210 and the plurality of tubes 4, in which mechanical connectors are not used for connecting the same. Each of the connector portions 210 have an internal flow path having an inner diameter and an outer surface having an outer diameter. The internal flow path of the connector portions 210 are complementary in configuration with the outer surface of the tube 4 such that the tube(s) 4 are able to be at least partly disposed within the connector portion(s) 210. Thus, the connector portions 210 of the connector 6 are fluidly connected to each other and the plurality of tubes 4 through the center 215 of the connector 6. It is appreciated that while in this embodiment, the connector 6 has an internal flow path that has an inner diameter that is equal to or similar to an inner diameter of the tube 4, the connector 6 can also have an internal flow path that has a larger or smaller inner diameter than the inner diameter of the tube depending on the requirements for transferring the pharmaceutical or biological fluid. For example, in an embodiment, if a central connector 6 is used to feed the pharmaceutical or biological fluid to the spine assembly, the central connector 6 can have an internal flow path with a larger inner diameter for distributing the pharmaceutical or biological fluid to the other connectors 6 of the spine assemblies that have a connector 6 and/or tubing 4 that has a smaller inner diameter. That is, the sizing of at least the inner diameter of the connector and/or the tube is volume dependent, e.g., the connecter can be sized to be used as a reducer, expander, or combination thereof.

The connector portion 210 continuously formed with the tube 4 can be performed in a variety of manners. For example, in an embodiment, the connector portion 210 is overmolded with the tubing 4 in a single overmolding process, e.g., using a single mold during the molding process (e.g., heat curing or using injection molding), cast molding (e.g., two-part cast molding or the like), thermoforming, or the like. Accordingly, the connector portion 210 and the tube 4 are formed directly with or continuously formed of polymer with each other. In other embodiments, it is appreciated that the connector portion 210 and the tube 4 can be continuously formed using other processes, for example, using welding or bonding techniques which can include, but are not limited to heat bonding, impulse welding, laser welding, ultrasonic welding, platen welding, or similar fusion bonding/melt welding techniques. That is, it is appreciated that the continuous formation of the connector portion and the tube results in a connector assembly that is formed as a single piece, e.g., does not include intermediary non-continuously formed connections or use clamps between the connector portion(s) and the tube, by using bonding, fusing, or molding processes so that the tube is not able to be pulled out from the connector portion. Thus, the connector assembly and the tube have a sufficiently high pull out force so that no leakage points exist between the connector portion and the tube, even during cryogenic freezing.

That is, not wishing to be bound by theory, it was surprisingly found that by having the continuous formation or having the continuous formation of polymer of the connector portions with the tubes, potential leak points are eliminated since no connectors or clamps are provided to attach the tube to the connector portions of the connector. In so doing, at least because the fluid connection assembly is continuously formed, during the cryogenic freezing process, e.g., a temperature at −190° C. or lower, the fluid connection system does not have leak points and instead has an uninterrupted flow path from the process equipment through the fluid connection system, so that the fluid connection system is able to maintain structural integrity and prevent the ingress of the cryogenic fluid, e.g., contamination of the pharmaceutical or biological fluid. It was surprisingly found that when the connector and the tubes are formed from the same or similar polymers, e.g., a EVA, the connector and tubes have the same or similar coefficients of thermal contraction and/or thermal properties and/or material properties, e.g., rigidity. Thus, at least because the connector and the tubes contract at the same rate, e.g., have the same or similar thermal properties, potential leak points are eliminated so that the structural integrity of the system is able to be maintained.

It is appreciated that while the material of the connector 6 provides the connector with flexibility, the flexibility of the connector 6 can also be increased based on the structure of the connector 6. For example, the connector portions 210 are conically tapered, in which an outer diameter of the first end 212 of the connector portion 210 is larger than an outer diameter of the second end 214 of the connector portion 210. Thus, by having such a structure, the flexibility of the connector portions 210 is further increased.

As seen in FIG. 2C, the connector 6 can also be provided with additional strain relief portions to further increase the flexibility of the connector 6. For example, each connector portion 210 can include a plurality of rib sections 230 that are provided along the outer surface of the conically tapered connector portion 210. The plurality of rib sections 230 is provided in parallel arrangement along a length direction of the connector portion, in which the rib section provided closest or nearest the center 215 of the connector 6 at the first end 212 has an outer diameter that is larger than a rib section provided at the second end 214 of the connector portion connected to the tube 4. It is appreciated that while FIG. 2C includes four rib sections, the number and placement of rib sections can be varied depending on the size/length of the connector, tube, and various other factors for the design of the connector when used for the transfer of the fluid, e.g., rib sections can be spaced further apart or closer together. By having such a structure, the connector portions 210 of the connector 6 are able to be further articulated, e.g., moved, so that when a tube is moved or adjusted, the connector portion 210 is articulated and/or bent so that the tube 4 is not stressed which prevents the formation of restrictions in the flow path. That is, since kinks in the tube and/or the pinching or buckling of the tube are not created, the flow transition between the connector and tubes is maintained, e.g., maintains a smooth flow curvature along the flow path.

FIGS. 2C and 2D also show that the connector 6 can also be provided with portions that are removed around the center 215 or has a center 215 that is thinner than the connector portions 210. That is, as seen in FIG. 2D, the center 215 has a center portion 216 in which material of the connector 6 has been removed so that the center portion 216 has a thinner wall thickness than the remainder of the connector 6, in which the center portion has a rectangular shape, e.g., cubical profile. It is appreciated that the center portion 216 can also have other geometric shapes, such as conical, cylindrical, or similar shape. By having the center portion 216 of the connector 6 have a thinner wall thickness and/or have material removed from the center portion, flexibility of the connector 6 is further increased so that the connector portions 210 are able to be further articulated with the moving of the tubes so that the flow transitions between the tube and the connector portion remain unrestricted, e.g., no kinking or pinching or buckling of the tube when the tube is moved.

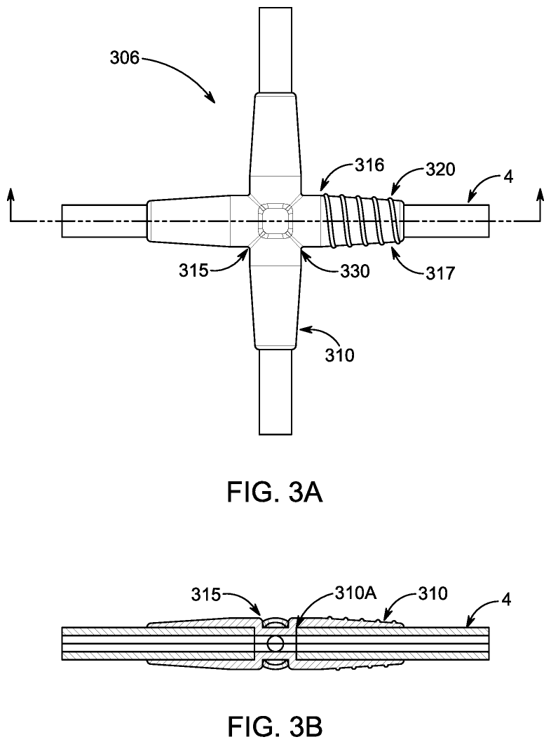

It is appreciated that the strain relief portions can include other structures to increase the flexibility of the connector 6. FIG. 3A illustrates an embodiment to increase flexibility of the connector 306 that includes strain relief portions in which the outer surface of the connector portion 310 includes spiral rib section 320 provided along a length direction on the outer surface of the connector portion. The spiral rib section 320 has a first end 316 provided nearer the center 315 of the connector 306 that has an outer diameter larger than the spiral rib section 310 at a second end 317 provided at the second end 314 of the connector portion 310. While a thickness of the spiral rib section is shown in FIG. 3A, it is appreciated that the thickness of the spiral rib section can vary to impart flexibility of the connector portion. Additionally, in some embodiments, as seen in FIG. 3A, the center 315 of the connector 306 can include segmented core portions 330 provided between adjacent connector portions 310, in which material around the center 315 is removed to further increase flexibility of the connector portions 310, e.g., the connector portions 310 can be further articulated when the tubes 4 are moved.

FIG. 3B shows a cross-sectional view of the connector 306 of FIG. 3A. As seen in FIG. 3B, in an embodiment, the connector 306 and the tube 4 can have the same or similar inner diameter, in which an outer surface of the tube 4 is complementary with the inner surface of the connector portion 310. An abutment surface 310A can be provided internally in the connector 306 between the center 315 and the connector portion 310, in which the tube 4 is abutted against the abutment surface 310A to enable alignment before the continuous formation of the connector and the tube. It is appreciated that the inner diameter of the connector 306 and tube 4 can also be sized so that the tube 4 can have a larger or smaller inner diameter depending on the requirements of the fluid transfer. That is, the sizing of at least the inner diameter of the connector and/or the tube is volume dependent, e.g., the connecter can be sized to be used as a reducer, expander, or combination thereof.

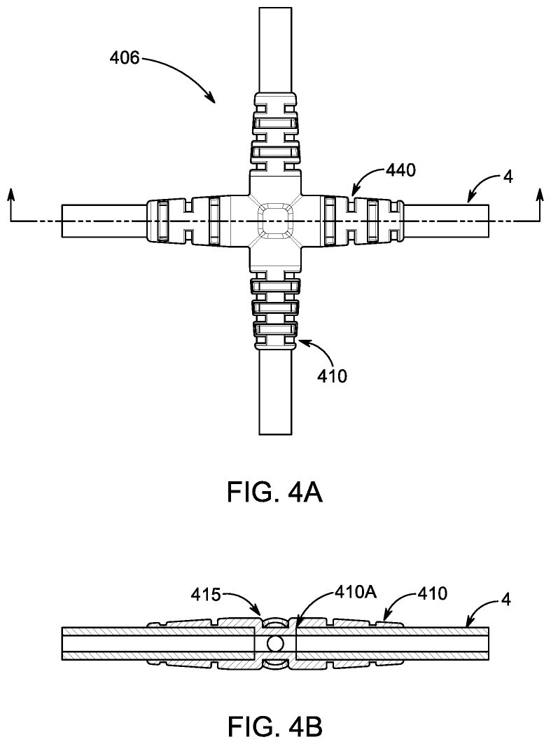

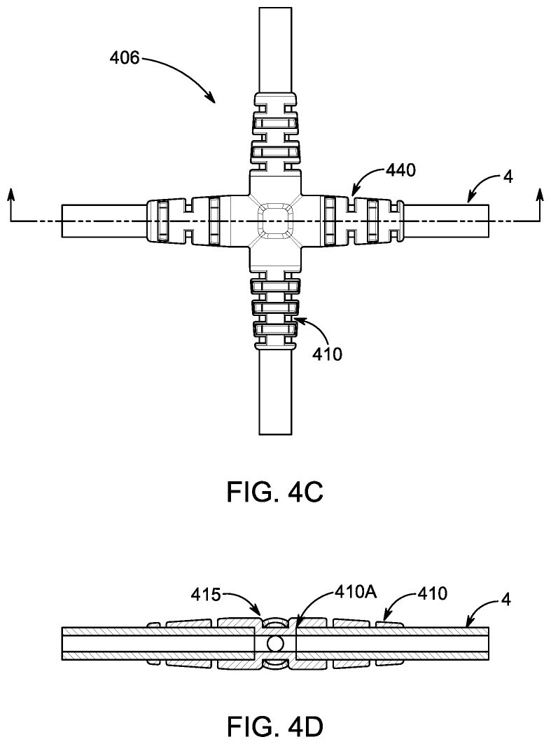

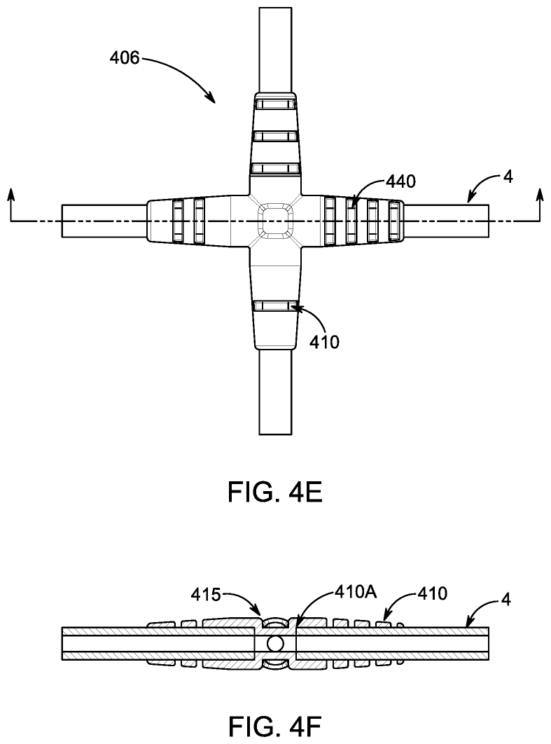

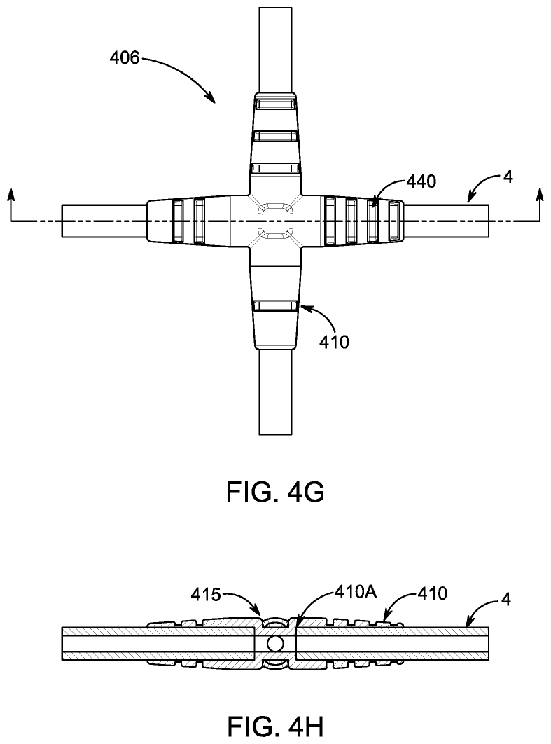

FIG. 4A illustrates another embodiment having strain relief portions to increase flexibility of the connector 406. In this embodiment, the strain relief portions of the connector portions 410 include segmented portions 440 removed from an outer surface of the connector portion 410. That is, the segmented portions are the remaining parts of the connector portion in which the outer surface is removed, so that the segmented portions result in the connector portion having a thickness equal to or the same as the nominal thickness of the connector portion. For example, the remaining thickness of the connector portion at the segmented portion can be 10-60% of the nominal wall thickness, and preferably a 50% thickness, and can be based on additional variables, such as, tubing flexibility (durometer), tubing characteristics during flex, OM resins used, connector geometry, e.g., has a sufficient thickness to maintain strength and stability of the connector portion 410, for example, having a thickness to withstand between 20-100 psi system pressure, and preferably about 30 psi system pressure and/or a pressure burst of between 100-300 psi. The segmented portions 440 can be provided on opposite sides of the connector portion 410 or alternately along a length of the connector portion in a way such that the flexibility of the connector portion 410 is increased. It is appreciated, as seen in FIG. 4A, that the number of segmented portions 440 can be varied, e.g., between 1 and 4, and the design can be varied, e.g., portions on opposite sides removed or entire sections are removed, along the length of the connection portions in order to increase flexibility of the connector portion, and any combinations thereof.

FIG. 4B shows a cross-sectional view of the connector 406 of FIG. 4A. As seen in FIG. 4B, in an embodiment, the connector 406 and the tube 4 can have the same or similar inner diameter, in which an outer surface of the tube 4 is complementary with the inner surface of the connector portion 410. An abutment surface 410A can be provided internally in the connector 406 between the center 415 and the connector portion 410, in which the tube 4 is abutted against the abutment surface 410A to enable alignment before the continuous formation of the connector and the tube.

FIG. 4C illustrates an alternative design of the connector 406 that includes the segmented portions 440. In this embodiment, the segmented portions 440 are portions of the connector portion that has been removed from the connector portion 410 so that the segmented portions 440 extend through the connector portion 410, e.g., the outer surface of the tube is exposed through the connector portion 410 at least through one or more of the segmented portions 440. It is appreciated that such design not only provides flexible strain relief but also allows retaining the tube in certain positions during the continuous formation of the connector and the tube.

FIG. 4D shows a cross-sectional view of the connector 406 of FIG. 4C. As seen in FIG. 4D, in an embodiment, the connector 406 and the tube 4 can have the same or similar inner diameter, in which an outer surface of the tube 4 is complementary with the inner surface of the connector portion 410. An abutment surface 410A can be provided internally in the connector 406 between the center 415 and the connector portion 410, in which the tube 4 is abutted against the abutment surface 410A to enable alignment before the continuous formation of the connector and the tube.

FIG. 4E illustrates another design of the connector 406 that includes the segmented portions 440. In this embodiment, the segmented portions 440 are also portions of the connector portion that have been removed from the connector portion 410 so that the segmented portions 440 extend through the connector portion 410, e.g., the outer surface of the tube is exposed through the connector portion 410 at least one or more of the segmented portions 440. In this embodiment, the segmented portions 440 are provided in a parallel arrangement along the length of the connector portion 410. It is appreciated that such design not only provides flexible strain relief but also allows retaining the tube in certain positions during the continuous formation of the connector and the tube.

FIG. 4F shows a cross-sectional view of the connector 406 of FIG. 4E. As seen in FIG. 4F, in an embodiment, the connector 406 and the tube 4 can have the same or similar inner diameter, in which an outer surface of the tube 4 is complementary with the inner surface of the connector portion 410. An abutment surface 410A can be provided internally in the connector 406 between the center 415 and the connector portion 410, in which the tube 4 is abutted against the abutment surface 410A to enable alignment before the continuous formation of the connector and the tube.

FIG. 4G illustrates another design of the connector 406 that includes the segmented portions 440. In this embodiment, the segmented portions 440 are provided in a parallel arrangement along the length of the connector portion 410 and are the remaining portion of the connector portion removed from an outer surface of the connector portion 410. That is, the segmented portions 410 are the remaining parts of the connector portion in which the outer surface is removed, so that the segmented portions result in the connector portion having a thickness equal to or the same as the nominal thickness of the connector portion. For example, the remaining thickness of the connector portion at the segmented portion can be 10-60% of the nominal wall thickness, and preferably has 50% of the thickness, and can be based on additional variables, such as, tubing flexibility (durometer), tubing characteristics during flex, OM resins used, connector geometry, e.g., has a sufficient thickness to maintain strength and stability of the connector portion 410, for example, having a thickness to withstand between 20-100 psi system pressure, and preferably about 30 psi system pressure and/or a pressure burst of between 100-300 psi.

FIG. 4H shows a cross-sectional view of the connector 406 of FIG. 4G. As seen in FIG. 4H, in an embodiment, the connector 406 and the tube 4 can have the same or similar inner diameter, in which an outer surface of the tube 4 is complementary with the inner surface of the connector portion 410. An abutment surface 410A can be provided internally in the connector 406 between the center 415 and the connector portion 410, in which the tube 4 is abutted against the abutment surface 410A to enable alignment before the continuous formation of the connector and the tube.

It is understood that the segmented portions 440 shown in FIGS. 4A-4H that are removed or partially removed can be various design and combined in various combinations to provide the strain relief for the connector portions without departing from the scope of the various embodiments.

It is also appreciated that while the designs of the connector have been discussed with respect to the strain relief portions on the outer surfaces of the connector portions, the various features can also at least partly extend through to an inner surface of the connector portion, e.g., extend through the wall thickness. Thus, at least a part of the rib, spiral, and/or segments portions extends into the inner surface so that the part of the rib, spiral, and/or segments portions can intersect with the tube inserted into the connector portion to maintain the position of the tube during the molding process.

While the above embodiments show the connector having four connector portions provided as a four-arm cross design, it appreciated that the connector can be provided in different design configurations within the various embodiments dependent on the number of the plurality of connector portions. For example, the connector can include a plurality of connector portions arranged as any combination of a four-arm cross mold design, a six-arm cross mold design (or star mold design), a tee-mold design, a y-mold design, an elbow mold design, or combination thereof. The connector design configuration can be chosen based on the number of bag assemblies required to be filled, layouts of the bag assemblies or processing equipment, or other design consideration. For example, the connector can be configured as a reducer, expander, or combination thereof when needed to connect different size tubing or components.

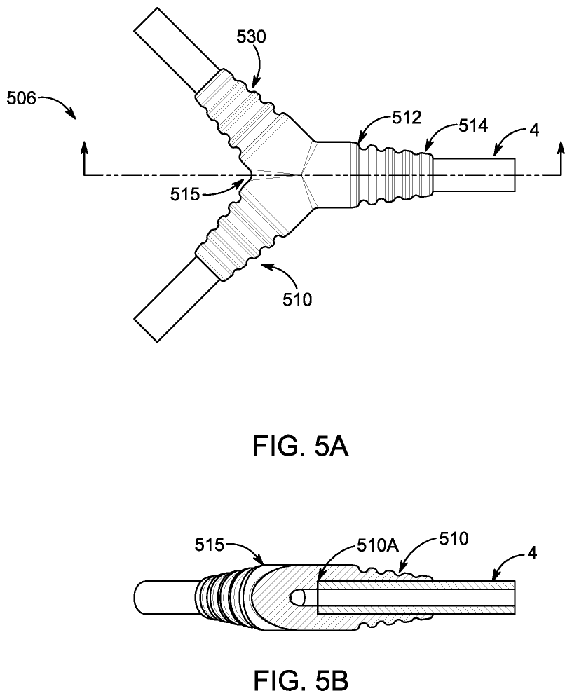

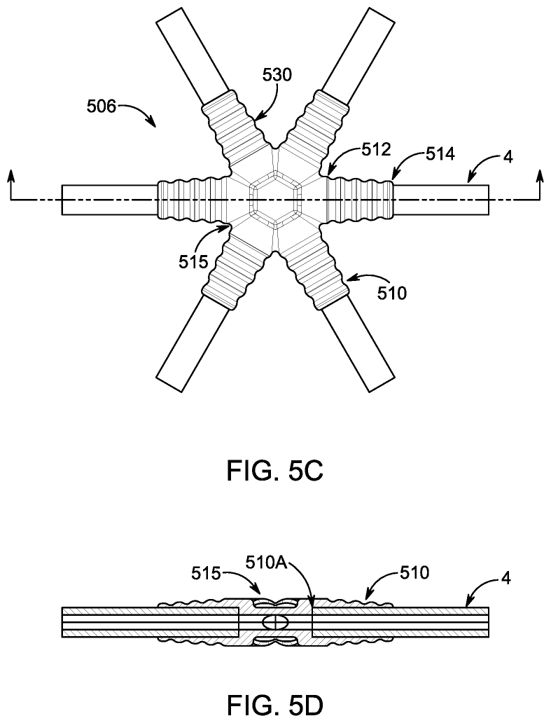

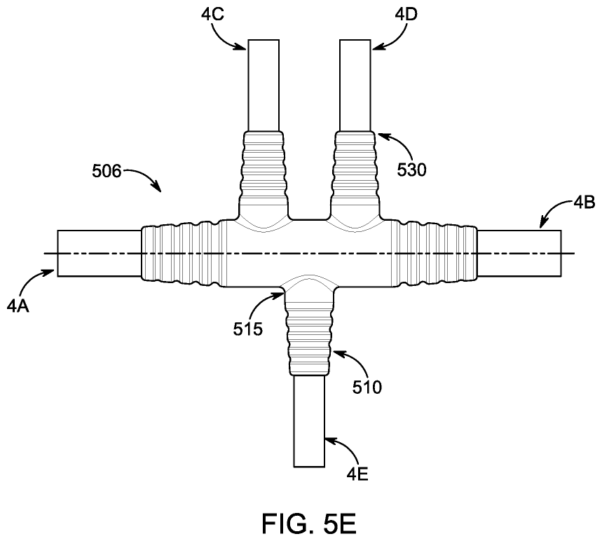

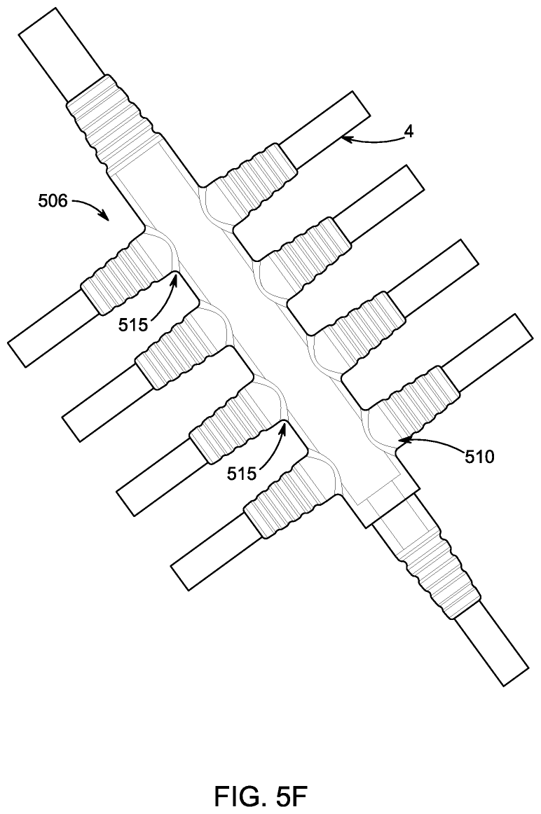

For example, as seen in FIG. 5A, the connector 506 includes three connector portions 510 in a y-mold design. In this embodiment, the connector portions 510 extend from the center 515 of the connector 506 in a y-design in which one of the tubes 4 can be provided for supplying the pharmaceutical or biological fluids to the other two connector portions 510. The connector portions 510 includes a plurality of rib sections 530 provided along a length of the outer surface of the connector portions 510 in parallel, in which the rib section provided closest or nearest the center 515 of the connector 506 at the first end 512 has an outer diameter that is larger than a rib section provided at the second end 514 of the connector portion connected to the tube 4.

FIG. 5B shows a cross-sectional view of the connector 506 of FIG. 5A. As seen in FIG. 5B, in an embodiment, the connector 506 and the tube 4 can have the same or similar inner diameter, in which an outer surface of the tube 4 is complementary with the inner surface of the connector portion 510. An abutment surface 510A can be provided internally in the connector 506 between the center 515 and the connector portion 510, in which the tube 4 is abutted against the abutment surface 510A to enable alignment before the continuous formation of the connector and the tube.

FIG. 5C illustrates another design of the connector portion, in which six connector portions 510 are provided as a six-arm cross mold design (or star mold design). In this embodiment, the connector portions 510 extend from the center 515 of the connector 506 in a six-arm cross or star-design in which one (or a plurality) of the tubes 4 can be provided for supplying the pharmaceutical or biological fluids to the other connector portions 510. The connector portions 510 includes a plurality of rib sections 530 provided along a length of the outer surface of the connector portions 510 in parallel, in which the rib section provided closest or nearest the center 515 of the connector 506 at the first end 512 has an outer diameter that is larger than a rib section provided at the second end 514 of the connector portion connected to the tube 4.

FIG. 5D shows a cross-sectional view of the connector 506 of FIG. 5C. As seen in FIG. 5D, in an embodiment, the connector 506 and the tube 4 can have the same or similar inner diameter, in which an outer surface of the tube 4 is complementary with the inner surface of the connector portion 510. An abutment surface 510A can be provided internally in the connector 506 between the center 515 and the connector portion 510, in which the tube 4 is abutted against the abutment surface 510A to enable alignment before the continuous formation of the connector and the tube.

FIG. 5E illustrates another design of the connector portion, in which five connector portions 510 are provided as a multiple-port design. In this embodiment, the connector portions 510 extend from the center 515 of the connector 506 in which the connector portions connected to the supply tubes have larger inner diameters than the remaining connector portions 510. For example, the tubes 4A and 4B provided horizontally in FIG. 5E can be used for supplying the pharmaceutical or biological fluids to the bag assemblies through the tubes 4C, 4D, 4E. In an embodiment, the connector portions 510 can have sizes that are similar to the size of the tubes, e.g., connector portions 510 connected to tubes 4A and 4B have larger inner and/or outer diameters than the remaining connector portions 510 of the connector 506. In this way, the connector 506 may be used as an expander, reducer, or combination thereof. Similar to the above embodiments, the connector portions 510 includes a plurality of rib sections 530 provided along a length of the outer surface of the connector portions 510 in parallel, in which the rib section provided closest or nearest the center 515 of the connector 506 at the first end 512 has an outer diameter that is larger than a rib section provided at the second end 514 of the connector portion connected to the tube 4. Thus, the rib sections provided at the connection portions 510 connected to tubes 4A and 4B have an outer diameter that is larger than the rib section(s) on the connection portions 510 connected to tubes 4C, 4D, 4E.

It is appreciated that the connector can include a single design configuration, or can be a combination of different design configurations, e.g., the different features of the various embodiments of the connectors can be combined with other designs or the same design of the connector. For example, as seen in FIG. 5F, the spine assembly 500 can include a connector 506 that that uses any combination of design configurations for the distribution of the pharmaceutical or biological fluid to the bag assemblies, e.g., the mold can include four four-arm cross mold designs for forming the connector. The connector 506 has a plurality of connector portions 510 that are conically tapered, where an outer diameter of the first end 512 at a plurality of centers 515 of the connector 506 is larger than an outer diameter of the second end 514. Thus, each of the connector portions 510 are flexible and able to be articulated, for example, when the bag assemblies are moved so that a flow restriction, e.g., kink or pinch, is not created in the flow transition between the tube 4 and the connector portions 510. It is also appreciated the flexibility of the connector portions can also reduce catch or snag points during movement of the bag assemblies (and tubing) and/or during packaging the system.

That is, all of the embodiments of the connector 6, 306, 406, 506 are formed having a structure that overcomes the deficiencies of prior art connector designs. For example, since the connector includes connector portions that are continuously formed with the tubes, the tubes and connector portions do not require mechanical connectors for connecting the same and no gaps between the tubing and the connector are created along the internal flow path. Thus, the structure of the connector avoids the creation of pockets that accumulate fluid and lead to contamination and the breakage of cells, e.g., the internal flow path is smooth, while maintaining a sufficient pull out force. Furthermore, it was surprisingly found, in some embodiments, that since the connector and tubes are continuously formed, if the connector and the pipe are made of the same or similar material, e.g., have the same or similar thermal properties, the fluid connection assembly can be used in cryogenic processes while maintaining the structural integrity of the system, e.g., leakage and/or contamination is prevented. For example, the connector is able to withstand an impact test at −196° C., freeze-drop test at −195° C., and be used for freeze-thaw applications.

Additionally, since the connector 6, 306, 406, 506 is flexible and the connector portions are able to be articulated when the tubes are moved, e.g., if a bag assembly or processing equipment is moved, the tube is not bent, e.g., not kinked or pinched, so that an interruption in the flow path between the flow transition between the tube and connector portion is not created. Thus, the fluid connection assembly is able to be used to transfer uniform amounts of the pharmaceutical or biological fluids to the bag assemblies, e.g., consistent flow can be maintained across the fluid connection assembly since no flow interruptions are created.

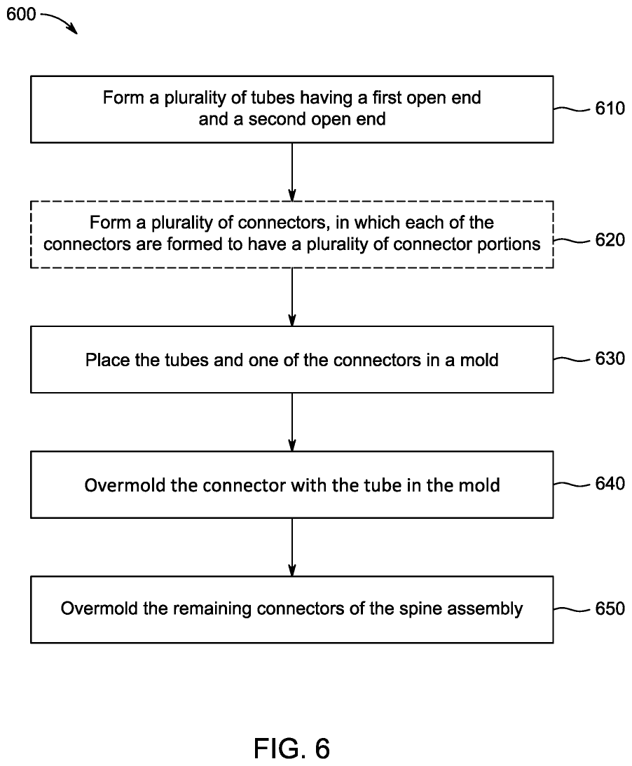

Referring to FIG. 6, a flow diagram for a method 600 of manufacturing a fluid connection assembly is depicted. For example, the method 600 may be used to manufacture the fluid connection assemblies in FIGS. 1-5F. Starting at S610, a plurality of tubes is formed having a first open end and a second open end opposite the first open end. At S620, a plurality of connectors is formed, in which each of the connectors are formed to have a plurality of connector portions. The connector portions are formed to have a first end connected to a center of the connector and a second open end. Each of the connector portions are formed to be conically tapered where an outer diameter of the first end at the center of the connector is larger than an outer diameter of the second end of the connector portion in a way such that the connector portion is flexible.

At S630, a plurality of tubes, e.g., at least three tubes, and one of the connectors are placed in a mold. It is appreciated that the tube(s) is inserted into the connector portion of the connector in a way such that mechanical friction holds the connector portion with the tube. For example, an open end of the tube can be inserted into the second end of the connector portion until it abuts an internal surface provided along an inner flow path of the connector portion or is inserted to a point that is at least one-quarter, and preferably one-half, the length of the connector portion. Thus, the tube is fluidly connected with the center of the connector and the other connector portions of the connector.

At S640, the connector is overmolded with the tube in the mold. The overmolding process can occur as a single overmolding process, e.g., using a single mold during the molding process (e.g., heat curing or using injection molding), cast molding (e.g., two-part cast molding or the like), thermoforming, or the like. For example, when a thermoset material is used, the thermoset resin material, e.g., silicone, is placed in a mold pin along with a catalyst or another resin. The mold pin is then heated, e.g., set, to a controlled temperature or the thermoset resin material is cured to form the connector overmolded with the tube. Alternatively, injection molding can be used, where a thermoplastic is injected into the mold to overmold the connector portions with the tubes. Accordingly, the connector and the connector portions and the tubes are formed directly with or continuously formed of polymer with each other.

At S650, the process is repeated for each connector and tube assembly to form the spine assembly of the fluid connection assembly by moving the mold pin to the remaining connectors to continuously form the connector portions of the connector with the other tube assemblies, e.g., sequential overmolding.

Aspects:

Any of aspects 1-9 can be combined with any of aspects 10-16 and/or 17-18 or vice-versa.

Aspect 1. A fluid connection assembly comprising:
a plurality of tubes having a first open end and a second open end opposite the first open end;
a plurality of connectors, each of the connectors comprising at least two connector portions, wherein each of the at least two connector portions extend from a center of the connector and have a first end connected to the center of the connector and a second end connected to the second open end of one of the plurality of tubes, wherein the connector portions are continuously formed with the tube and the center of the connector and wherein the connector portions are fluidly connected together,
wherein each of the at least two connector portions are conically tapered where an outer diameter of the first end at the center of the connector is larger than an outer diameter of the second end of the connector portion in a way such that the connector portion is flexible.

Aspect 2. The fluid connection assembly of aspect 1, wherein the connector portions comprise strain relief portions provided along an outer surface of the respective connector portions.

Aspect 3. The fluid connection assembly of aspect 2, wherein the strain relief portions comprise a plurality of rib sections provided in a parallel arrangement along a length direction of the connector portion, wherein a rib section provided nearer the center of the connector has an outer diameter larger than a rib section provided at the second end of the connector portion.

Aspect 4. The fluid connection assembly of aspect 2, wherein the strain relief portions comprise spiral rib sections provided along a length direction of the connector portion, wherein a first end of the spiral rib section provided nearer the center of the connector is provided at the first end of the connector portion having an outer diameter larger than a second end of the spiral rib section provided at the second end of the connector portion.

Aspect 5. The fluid connection assembly of aspect 2, wherein the strain relief portions comprise segmented core portions removed from the outer surface of the connector portion.

Aspect 6. The fluid connection assembly of aspect 2, wherein the connector comprises a thermoplastic elastomer or thermoset material.

Aspect 7. The fluid connection assembly of aspect 6, wherein the thermoplastic elastomer is ethylene-vinyl acetate (EVA) or silicon.

Aspect 8. The fluid connection assembly of any of aspects 1-7, wherein the connector has a design selected from a group consisting of a four-arm cross mold, a six-arm star mold, a tee mold, a y mold, an elbow mold, as a reducer, and combinations thereof.

Aspect 9. The fluid connection assembly of aspects 1-8, wherein the first end open end of one of the plurality of tubes is fluidly connected to a bag assembly.

Aspect 10. A connector comprising:
at least two connector portions, wherein each of the at least two connector portions extend from a center of the connector and have a first end connected to the center of the connector and a second end extending transversely from the center of the connector, wherein the connector portions are continuously formed with the center of the connector and wherein the connector portions are fluidly connected together,
wherein each of the at least two connector portions are conically tapered where an outer diameter of the first end at the center of the connector is larger than an outer diameter of the second end of the connector portion in a way such that the connector portion is flexible.

Aspect 11. The connector of aspect 10, wherein the connector portions comprise strain relief portions provided along an outer surface of the respective connector portions.

Aspect 12. The connector of aspect 11, wherein the strain relief portions comprise a plurality of rib sections provided in a parallel arrangement along a length direction of the connector portion, wherein a rib section provided nearer the center of the connector has an outer diameter larger than a rib section provided at the second end of the connector portion.

Aspect 13. The connector of aspect 11, wherein the strain relief portions comprise spiral rib sections provided along a length direction of the connector portion, wherein a first end of the spiral rib section provided nearer the center of the connector is provided at the first end of the connector portion having an outer diameter larger than a second end of the spiral rib section provided at the second end of the connector portion.

Aspect 14. The connector of aspect 11, wherein the strain relief portions comprise block core portions removed from the outer surface of the connector portion.

Aspect 15. The connector of any of aspects 10-14, wherein the connector comprises a thermoplastic elastomer.

Aspect 16. The connector of aspect 15, wherein the connector comprises thermoplastic ethylene-vinyl acetate (EVA).

Aspect 17. A method for manufacturing a fluid connection assembly comprising:
forming a plurality of tubes having a first open end and a second open end opposite the first open end;
forming a plurality of connectors, wherein each of the connectors is formed to have at least two connector portions, wherein each of the at least two connector portions extend from a center of the connector and have a first end connected to the center of the connector and a second end connected to the second open end of one of the plurality of tubes, wherein the connector portions are fluidly connected together;
overmolding at least one of the two connector portions with the second open end of one of the plurality of tubes so that the connector portion is fluidly connected with the center of the connector and the tube,
wherein each of the at least two connector portions are formed to be conically tapered where an outer diameter of the first end at the center of the connector is larger than an outer diameter of the second end of the connector portion in a way such that the connector portion is flexible.

Aspect 18. The method of aspect 17, wherein the overmolding step occurs in a single step.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

What is claimed is:

1. A fluid connection assembly comprising:
a plurality of tubes having a first open end and a second open end opposite the first open end;
a plurality of connectors, each of the connectors comprising at least two connector portions, wherein each of the at least two connector portions extend from a center of the connector and have a first end connected to the center of the connector and a second end connected to the second open end of one of the plurality of tubes, wherein the connector portions are continuously formed with the tube and the center of the connector and wherein the connector portions are fluidly connected together,
wherein each of the at least two connector portions are conically tapered where an outer diameter of the first end at the center of the connector is larger than an outer diameter of the second end of the connector portion in a way such that the connector portion is flexible.

2. The fluid connection assembly according to claim 1, wherein the connector portions comprise strain relief portions provided along an outer surface of the respective connector portions.

3. The fluid connection assembly according to claim 2, wherein the strain relief portions comprise a plurality of rib sections provided in a parallel arrangement along a length direction of the connector portion, wherein a rib section provided nearer the center of the connector has an outer diameter larger than a rib section provided at the second end of the connector portion.

4. The fluid connection assembly according to claim 2, wherein the strain relief portions comprise spiral rib sections provided along a length direction of the connector portion, wherein a first end of the spiral rib section provided nearer the center of the connector is provided at the first end of the connector portion having an outer diameter larger than a second end of the spiral rib section provided at the second end of the connector portion.

5. The fluid connection assembly according to claim 2, wherein the strain relief portions comprise segmented core portions removed from the outer surface of the connector portion.

6. The fluid connection assembly according to claim 1, wherein the connector comprises a thermoplastic elastomer or thermoset material.

7. The fluid connection assembly according to claim 6, wherein the thermoplastic elastomer is ethylene-vinyl acetate (EVA) or silicon.

8. The fluid connection assembly according to claim 1, wherein the connector has a design selected from a group consisting of a four-arm cross mold, a six-arm star mold, a tee mold, a y mold, an elbow mold, as a reducer, and combinations thereof.

9. The fluid connection assembly according to claim 1, wherein the first end open end of one of the plurality of tubes is fluidly connected to a bag assembly.

10. A connector comprising:
at least two connector portions, wherein each of the at least two connector portions extend from a center of the connector and have a first end connected to the center of the connector and a second end extending transversely from the center of the connector, wherein the connector portions are continuously formed with the center of the connector and wherein the connector portions are fluidly connected together,
wherein each of the at least two connector portions are conically tapered where an outer diameter of the first end at the center of the connector is larger than an outer diameter of the second end of the connector portion in a way such that the connector portion is flexible.

11. The connector according to claim 10, wherein the connector portions comprise strain relief portions provided along an outer surface of the respective connector portions.

12. The connector according to claim 11, wherein the strain relief portions comprise a plurality of rib sections provided in a parallel arrangement along a length direction of the connector portion, wherein a rib section provided nearer the center of the connector has an outer diameter larger than a rib section provided at the second end of the connector portion.

13. The connector according to claim 11, wherein the strain relief portions comprise spiral rib sections provided along a length direction of the connector portion, wherein a first end of the spiral rib section provided nearer the center of the connector is provided at the first end of the connector portion having an outer diameter larger than a second end of the spiral rib section provided at the second end of the connector portion.

14. The connector according to claim 11, wherein the strain relief portions comprise block core portions removed from the outer surface of the connector portion.

15. The connector according to claim 10, wherein the connector comprises a thermoplastic elastomer.

16. The connector according to claim 15, wherein the connector comprises thermoplastic ethylene-vinyl acetate (EVA).

17. A method for manufacturing a fluid connection assembly comprising:
forming a plurality of tubes having a first open end and a second open end opposite the first open end;
forming a plurality of connectors, wherein each of the connectors is formed to have at least two connector portions, wherein each of the at least two connector portions extend from a center of the connector and have a first end connected to the center of the connector and a second end connected to the second open end of one of the plurality of tubes, wherein the connector portions are fluidly connected together;
overmolding at least one of the two connector portions with the second open end of one of the plurality of tubes so that the connector portion is fluidly connected with the center of the connector and the tube,
wherein each of the at least two connector portions are formed to be conically tapered where an outer diameter of the first end at the center of the connector is larger than an outer diameter of the second end of the connector portion in a way such that the connector portion is flexible.

18. The method according to claim 17, wherein the overmolding step occurs in a single step.
